(19) United States
(12) Patent Application Publication
(10) Pub. No.: US 2005/0026152 A1
(43) Pub. Date: Feb. 3, 2005

(54) Methods of screening for schizophrenia

(76) Inventor: Norbert Muller, Munchen (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/624,570

(22) Filed: Jul. 23, 2003

Related U.S. Application Data
(60) Provisional application No. 60/397,611, filed on Jul. 23, 2002.

(51) Int. Cl. C12Q 1/68; C12P 19/34

(52) U.S. Cl. 435/6; 435/91.2

(57) ABSTRACT

A novel association between a polymorphism at amino acid residue 241 of ICAM-1 and schizophrenia has been discovered. The present invention concerns methods of screening for schizophrenia and the use of kits which exploit the present invention.

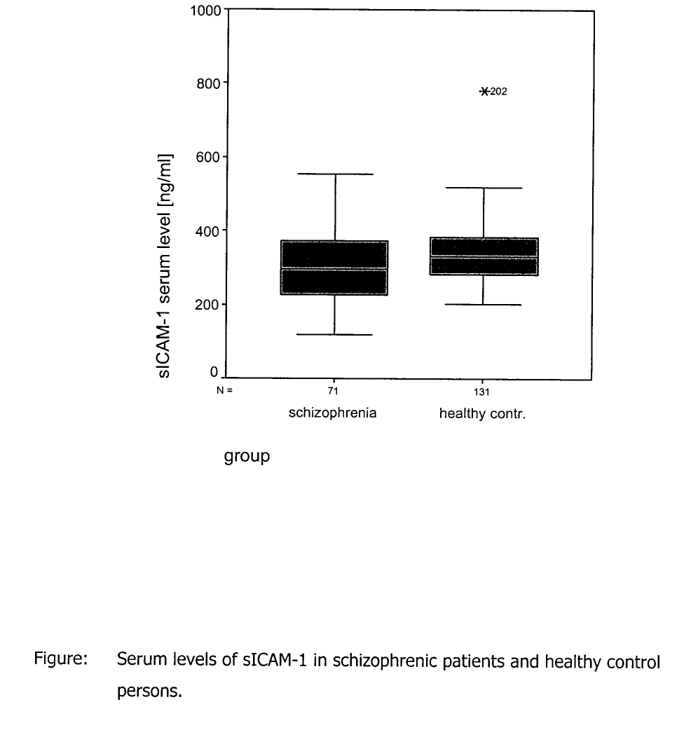

### FIELD OF THE INVENTION

The present invention relates to a method and kit for screening for schizophrenia. More specifically, it concerns a genetic polymorphism in intercellular adhesion molecule-1 (ICAM-1), which has been found to be associated with schizophrenia.

### BACKGROUND OF THE INVENTION

An important genetic contribution to the aetiology of schizophrenia is well established from genetic epidemiological studies: The risk of developing schizophrenia in family members increases with the degree of biological relatedness to the patient—greater risks are associated with higher levels of shared genes (Tsuang M T, Stone W S, Faraone S V. Genes, environment and schizophrenia. Br J Psychiatry Suppl 2001; 40: s18-s24). The mode of transmission like that of other common disorders seems to be complex and non-Mendelian (McGue M, Gottesman I I. A single dominant gene still cannot account for the transmission of schizophrenia. Arch Gen Psychiatry 1989; 46: 478-480). An oligogenic or polygenic mode of transmission is the most probable genetic basis of schizophrenia (McGuffin P, Owen M J, Farmer A E. Genetic basis of schizophrenia. Lancet 1995; 346: 678-682). The current choice of candidate genes for association studies evolved from the neurochemical hypothesis of schizophrenia, based on the actions of antipsychotic agents (Jurewicz I, Owen R J, O'Donovan M C, Owen M J. Searching for susceptibility genes in schizophrenia. Eur Neuropsychopharmacol 2001; 11: 395-398). However, other candidate genes may be considered on the basis of other biochemical findings.

Schizophrenia is a heterogenous disorder with a world-wide prevalence of about 1% and a high socio-economic impact. It is characterized by symptoms such as halluzinations or disorganized thinking, loss of goal-directed behaviours, and deterioration in social role functioning. Direct and indirect costs have been estimated 4.1 billion US $ per year (Knapp M., Cost of Schiophrenia, Brit. J. Psychiatry, 1995).

Although the last decades have brought tremendous progress in the diagnosis, classification, and treatment of schizophrenia, the etiology of the disease remains unknown. However, a hereditary component has to be assumed and there is major evidence for the involvement of an immune process in the pathophysiology of the disorder.

The involvement of an immune process in the pathophysiology of schizophrenia has repeatedly been hypothesised (Ganguli R, Brar I S, Chengappa K N, Yang Z W, Nimgaonkar V L, Rabin B S. Autoimmunity in schizophrenia: a review of recent findings. Ann Med 1993; 25: 489-496). In-vitro lymphocyte stimulation studies showed a reduced production of the pro-inflammatory, Th1-like cytokines Interferon-γ (IFN-γ) or Interleukin-2 (IL-2) in cells of schizophrenic patients (Arolt V, Rothermundt M, Wandinger K P, Kirchner H. Decreased in-vitro production of Interferon-γ and Interleukin-2 in whole blood of patients with schizophrenia during treatment. Mol Psychiatry 2000; 5: 150-158). Serum levels of soluble ICAM-1 was demonstrated to be reduced in unmedicated and medicated schizophrenic patients (Schwarz M J, Riedel M, Ackenheil M, Müller N. Decreased Levels of Soluble Intercellular Adhesion Molecule-1 (sICAM-1) in Unmedicated and Medicated Schizophrenic Patients. Biol Psychiat 2000; 47: 29-33). Elevated levels of antibody titers directed against a variety of (auto)antigens were found in serum and cerebrospinal fluid (CSF) of schizophrenic patients (Heath R G, McCarron K L, O'Neil C E. Antiseptal brain antibody in IgG of schizophrenic patients. Biol Psychiatry 1989; 25: 725-733; Sundin U, Thelander S. Antibody reactivity to brain membrane proteins in serum from schizophrenic patients. Brain Behav Immun 1989; 3: 345-358; Henneberg A E, Horter S, Ruffert S. Increased prevalence of antibrain antibodies in the sera from schizophrenic patients. Schizophr Res 1994; 14: 15-22; Schwarz M J, Riedel M, Gruber R, Ackenheil M, Müller N. Antibodies to Heat Shock Proteins in Schizophrenic Patients: Implications for the Mechanism of the Disease. Am J Psychiatry 1999; 156: 1103-1104). Based on these findings, several authors concluded a reduced T cell response in at least a subgroup of schizophrenic patients (Rothermundt M, Arolt V, Weitzsch C, Eckhoff D, Kirchner H. Immunological dysfunction in schizophrenia: a systematic approach. Neuropsychobiology 1998; 37: 186-193; Schwarz M J, Chiang S, Müller N, Ackenheil M. T-helper-1 and T-helper-2 responses in psychiatric disorders. Brain Behav Immun 2001; 15: 340-370). This reduced T cell response was interpreted as a reduced activation of the Th1 cells accompanied by a relatively more pronounced activity of Th2 cells (Schwarz M J, Chiang S, Müller N, Ackenheil M. T-helper-1 and T-helper-2 responses in psychiatric disorders. Brain Behav Immun 2001; 15: 340-370). However, the causative factor of these immunological abnormalities in schizophrenia has not been unravelled until now, but it is commonly accepted that a genetic predisposition has to interact with an environmental risk factor, e.g. a viral infection (Torrey E F, Yolken R H. Could schizophrenia be a viral zoonosis transmitted from house cats? Schizophr Bull 1995; 21: 167-171).

A balance exists in healthy CNS between the Th1- and the Th2-system, being represented in the CNS by microglia cells (predominantly Th1-function) and astrocytes (predominantly Th2-function). Moreover, lymphocytes penetrating from the peripheral immune system contribute to the priming and function of the CNS by co-activation. Penetration of lymphocytes from the peripheral immune system into the CNS occurs in significant amounts only when the lymphocytes were peripherically stimulated and an antigene presentation in the CNS occurs, thereby transmitting a signal to the lymphocytes. In the peripheral system monocytes and macrophages as antigene presenting cells are divided in M1 and M2 according to their function. M1 are mainly antigene presenting cells producing Th1-cytokines, whereas M2 are predominantly cytokines producing cells, activated via an alternative route, in case the M1-route is functionally blocked. Without the occurrence of an infection, this balance exists at low level. Possible dysfunctions are not manifested without a challenge of the system. However, in case a signal is not recognized such dysfunction may turn into a clinical pathological state. In case of a virus infection at first an activation of the cellular immune response with an early synthesis and secretion of IL-12 and ICAM-1 takes place, if a dysfunction is present, activation of the Th2-system by IL-10 occurs alternatively.

The two main functions of ICAM-1 are the following:
adhesion molecule responsible for the penetration of lymphocytes through the endothelium in the parenchym, in particular through the blood brain barrier
co-activator of the T cell system, especially of the Th1-system.

Activation of T cells may require a minimum of two signals by antigen-presenting cells such as macrophages and B cells: the first signal delivered by major histocompatibility complex (MHC)-peptide complex and a second signal, delivered by cell surface molecules such as the intercellular adhesion molecule-1 (ICAM-1) (Lamphear J G, Stevens K R, Rich R R. Intercellular adhesion molecule-1 and leukocyte function-associated antigen-3 provide costimulation for superantigene-induced T lymphocyte proliferation in the absence of a specific presenting molecule. J Immunol 1998; 160: 615-623). The adhesion molecule ICAM-1 is a transmembrane glycoprotein, which belongs to the immunoglobulin superfamily, and contains five tandem immunoglobulin-like domains. The two ligands of ICAM-1 are the integrins LFA-1 (lymphocyte function-associated antigen-1, CD11a/CD18) and MAC-1 (Macrophage 1 antigene, CD11b/CD18) (Hogg N, Leitinger B. Shape and shift changes related to the function of leukocyte integrins LFA-1 and Mac-1. J Leukoc Biol 2001; 69: 893-898). LFA-1 and Mac-1 are the binding sites for ICAM-1-mediated activation of lymphocytes and macrophages respectively.

Thus, a genetic variation in the ICAM-1 molecule would be an important variable in the susceptibility, diagnosis and treatment of psychiatric disorders such as schizophrenia which involve immunological responses.

### SUMMARY OF INVENTION

Based on the discovery of an association between schizophrenia and a polymorphism in ICAM-1, the present invention relates to a method of screening for schizophrenia, comprising the step of:
(a) assaying a nucleic acid sample for the presence of a codon encoding arginine at amino acid position 241 of the ICAM-1 protein,
   wherein the presence of a codon encoding arginine at position 241 of the ICAM-1 protein is indicative of schizophrenia. More specifically, the present invention relates to a method of screening for schizophrenia in male schizophrenic patients exhibiting a significantly different ICAM-1 241 genotype distribution compared to male control subjects. Disclosed herein is strong evidence of a functional immunological involvement of the ICAM-1 gene G241A polymorphism.

In another aspect of the present invention we report that serum concentrations of sICAM-1 in schizophrenic patients are significantly lower than in controls. In particular, a significant difference between the serum concentration of sICAM-1 in female patients and in female controls was observed.

According to one embodiment, the present invention is directed to a method of screening for schizophrenia where in addition to the ICAM-1 polymorphism a further polymorphism serves as marker for the susceptibility for a schizophrenia.

Also provided is the use of a kit for screening for psychiatric disorders comprising reagents tailored to identify the polymorphism at amino acid position 241 of the ICAM-1 protein.

In a further aspect the present invention concerns the use of a method of screening for schizophrenia for predicting clinical responses to a therapeutic compound in the treatment of ICAM-1 mediated schizophrenia.

Also provided are descriptions and claims to therapies related to and using modulators of the ICAM-1 polymorphism.

According to a further embodiment, the inventive method further comprises assaying said sample for an additional marker associated with the susceptibility for schizophrenia, said marker being the single nucleotide polymorphism G1188T IL-12 SNP (SNP). Other polymorphisms and associations are also disclosed.

### SHORT DESCRIPTION OF THE FIGURE

The FIGURE shows serum levels of sICAM-1 in schizophrenic patients and controls.

### DETAILED DESCRIPTION OF THE INVENTION

An association between schizophrenia and a polymorphism in ICAM-1 has been discovered. The inventors have indeed demonstrated that the presence of a codon encoding arginine at amino acid position 241 of the ICAM-1 protein is indicative of a schizophrenia. More particularly, it was found that this polymorphism is useful in the diagnosis of schizophrenia in male schizophrenic patients. This polymorphism can be exploited for providing convenient methods of screening for schizophrenia, enabling the diagnosis and determination of susceptibility to schizophrenia. Treatments for schizophrenia can now be custom tailored taking into account an individuals or a populations possession or prevalence of the 241 polymorphism.

The human ICAM-1 gene is a single copy gene located on chromosome 19, in the region 19p13.3-p13.2 (Vora D K, Rosenbloom C L, Beaudet A L, Cottingham R W. Polymorphisms and linkage analysis for ICAM-1 and the selectin gene cluster. Genomics 1994; 21: 473-477). The human ICAM-1 gene consists of seven exons, separated by six introns. Each of the five immunoglobulin (Ig)-like domains is encoded by a separate exon (Voraberger G, Schafer R, Stratowa C. Cloning of the human gene for intercellular adhesion molecule 1 and analysis of its 5′-regulatory region. Induction by cytokines and phorbol ester. J Immunol 1991; 147: 2777-2786). The ICAM-1 protein consists of 505 amino acids. The Ig-like domains are formed by 453 predominantly hydrophobic amino acids, followed by a hydrophobic transmembrane domain and a charged cytoplasmic tail (Staunton D E, Marlin S D, Stratowa C, Dustin M L, Springer T A. Primary structure of ICAM-1 demonstrates interaction between members of the immunoglobulin and integrin supergene families. Cell 1988; 52: 925-933). The binding site for LFA-1 is represented by the fifth Ig-like domain, while the third domain represents the Mac-1 binding site (Vora D K, Rosenbloom C L, Beaudet A L, Cottingham R W. Polymorphisms and linkage analysis for ICAM-1 and the selectin gene cluster. Genomics 1994; 21: 473-477). The complete nucleotide sequence of ICAM-1 gene is published in EMBL GenBank under accession number X59286, X59287 and X59288, all of which is incorporated herein by reference. The two most frequently investigated functional single nucleotide polymorphisms (SNPs) of the ICAM-1 gene are exactly located in these two domains: The G→A SNP at position 241 in exon 4, the Mac-1 binding domain (Vora D K, Rosenbloom C L, Beaudet A L, Cottingham R W. Polymorphisms and linkage analysis for ICAM-1 and the selectin gene cluster. Genomics 1994; 21: 473-477), and the A→G SNP at position 469 in exon 6, the LFA-I binding site (Joling P, Boom S, Johnson J, Dekker M E, van den Tweel J G, Schuurman H J, Bloem A C. Domain 5 of the intercellular adhesion molecule-1 (ICAM-1) is involved in adhesion of B-cells and follicular dendritic cells. Adv Exp Med Biol 1994; 355: 131-135). Both polymorphisms lead to an amino acid change in the ICAM-1 protein sequence (Gly [GGG] to Arg [AGG] in position 241 and Lys [AAG] to Glu [GAG] in position 469) (Vora D K, Rosenbloom C L, Beaudet A L, Cottingham R W. Polymorphisms and linkage analysis for ICAM-1 and the selectin gene cluster. Genomics 1994; 21: 473-477). A nucleic acid encoding a glycine at amino acid residue 241 of ICAM-1 is referred herein as the “G241 allele”, a nucleic acid encoding an arginine at amino acid residue 241 of ICAM-1 is referred herein as the “A241 allele” (polymorphic A-Allel). Significant differences in allele frequencies of both polymorphisms have been detected in several diseases like rheumatoid arthritis (RA) (Macchioni P, Boiardi L, Casali B, Nicoli D, Farnetti E, Salvarani C. Intercellular adhesion molecule 1 (ICAM-1) gene polymorphisms in Italian patients with rheumatoid arthritis. Clin Exp Rheumatol 2000; 18: 553-558), Behcet's disease (Boiardi L, Salvarani C, Casali B, Olivieri I, Ciancio G, Cantini F, Salvi F, Malatesta R, Govoni M, Trotta F, Filippini D, Paolazzi G, Nicoli D, Farnetti E, Macchioni L. Intercellular adhesion molecule-1 gene polymorphisms in Behcet's Disease. J Rheumatol 2001; 28: 1283-1287), multiple sclerosis (Mycko M P, Kwinkowski M, Tronczynska E, Szymanska B, Selmaj K W. Multiple sclerosis: the increased frequency of the ICAM-1 exon 6 gene point mutation genetic type K469. Ann Neurol 1998; 44: 70-75), and insuline-dependent diabetes mellitus (Nishimura M, Obayashi H, Maruya E, Ohta M, Tegoshi H, Fukui M, Hasegawa G, Shigeta H, Kitagawa Y, Nakano K, Saji H, Nakamura N. Association between type 1 diabetes age-at-onset and intercellular adhesion molecule-1 (ICAM-1) gene polymorphism. Hum Immunol 2000; 61: 507-510). U.S. Pat. No. 5,681,699 and U.S. Pat. No. 6,008,335 disclose an association between inflammatory Bowel disease and ICAM-1 polymorphism (G214A). This polymorphism provides the basis for methods of screening for IBD, methods for treating IBD and kits exploiting these methods.

In the present method of screening, the following step will be performed:
(a) assaying a nucleic acid sample for the presence of a codon encoding arginine at amino acid position 241 of the ICAM-1 protein,
   wherein the presence of a codon encoding arginine at position 241 of the ICAM-1 protein is indicative of schizophrenia.

According to the present invention, any biological samples which contain a nucleic acid sample, in particular genomic DNA, may be employed in step (a) of the present method, including tissue samples and blood samples, preferably the nucleic acid sample is isolated from blood cells. The term nucleic acid sample includes DNA, such as genomic DNA or cDNA, and RNA. According to a preferred embodiment, prior to step (a) there is a step of obtaining a nucleic acid sample from a patient.

The step (a) of assaying the nucleic acid sample for the presence of a single nucleotide polymorphism at amino acid position 241 of the ICAM-1 protein, namely the presence of a codon AGG encoding arginine, may be carried out by any suitable means.

A variety of techniques are known to those skilled in the art. All generally involve the step of amplification reaction such as a polymerase chain reaction (PCR) or ligase chain reaction. Amplification of a selected, or target, nucleic acid sequence may be carried out by any suitable means (see D. Kwoh and T. Kwoh, Am. Biotecnol. Lab. 8, 14-25 (1990)). Polymerase chain reaction is the preferred technique. Most preferably, a SNAPshot method is carried out comprising as first step a multiplex PCR and as the second step a SNAPshot PCR. (Informations about SNaPshot method can be found at the website of Applied Biosystems:
http://www.appliedbiosystems.com/products/productdetail.cfm?prod_id=502 and at the following site: http://www.genetics.med.ed.ac.uk/protocols/GGTSPU-12818-1534784-DAT/SNaPshot.pdf; and in a publication wherein the SNaPshot method has been used: Yamaguchi N, Kobayashi K, Yasuda T, Nishi I, Iijima M, Nakagawa M, Osame M, Kondo I, Saheki T (2002). Screening of SLC25A13 mutations in early and late onset patients with citrin deficiency and in the Japanese population: Identification of two novel mutations and establishment of multiple DNA diagnosis methods for nine mutations. Hum Mutat 2002 February;19(2):122-130.)

According to a preferred embodiment, a pair of primers having the sequence SEQ ID NO: 1 and SEQ ID NO: 2 is used for the amplification by PCR of the specific fragment of the ICAM-1 gene containing the G241A polymorphism. Preferably, a SNAPshot primer having the sequence SEQ ID NO: 3 is used in the SNAPshot PCR.

A PCR protocol for determining the codon at amino acid position 241 of the ICAM-1 protein is described in the experimental part of the present specification.

According to a further embodiment, the inventive method further comprises assaying said sample for an additional marker associated with the susceptibility for schizophrenia, said marker being the G1188T IL-12 single nucleotide polymorphism (SNP).

It has been found that a further polymorphism may be associated with the risk of developing schizophrenia, and this or further SNP's can be used, alone or in combination with the ICAM-1 polymorphism, for the assessment of an additional risk factor for developing schizophrenia. The IL-12 polymorphism may reduce the activation of M1 as precursor of the ICAM-1 coactivation. In fact, the production of IL-12 by dendritic cells and monocytes is crucial for Th1 differentiation (Gately M K, Renzetti L M, Magram J, Stern A S, Adorini L, Guber U, Presky D H (1998) The interleukin 12/interleugin 12 receptor system: role in normal and pathologic immune responses. Annu Rev Immunol 16: 495-521). Intracerebral IL-12 synthesis is induced early following viral infection and is thought to play an important role in establishing IFN-γ dependent resistance and polarisation of Th1-responses (Komatsu T, Barna M, Reiss C S (1997) Interleukin-12 promotes recovery from viral encephalitis. Viral Immunol 10: 35-47; Sato S, Reiner S L, Jensen M A, Roos R P (1997) Central nervous system cytokine mRNA expression following Theiler's murine encephalomyelitis virus infection. J Neuroimmunol 76: 213-223). The intracerebral IL-12 synthesis is an important event in sustaining Th1-responses. IL-12 sources in the inflamed CNS include cells of the immune infiltrate (mostly macrophages, if invaded) and resident CNS cells, particularly microglia.

In another aspect the present invention comprises the use of a kit for screening for schizophrenia comprising reagents tailored to identify the polymorphism at position 241 of the ICAM-1. Said reagents are selected from nucleotide probes that selectively bind to DNA encoding a specific fragment of the ICAM-1 protein including the polymorphic position 241 of the ICAM-1. Such reagents preferably comprise a pair of primers having the sequences SEQ ID NO: 1 and 2 and reagents necessary to perform the PCR. A detailed description of such a kit is disclosed in U.S. Pat. No. 6,008,335 and U.S. Pat. No. 5,681,699, incorporated herein by reference. Furthermore, the kit may comprise reagents tailored to identify the further marker respectively polymorphism, that is G1188T IL-12 SNP. Said reagents are selected from oligonucleotide probes that selectively bind to DNA encoding a specific fragment of the protein including the polymorphism. Such reagents preferably comprise a pair of primers having the sequences SEQ ID NO: 4 and 5 for the G1188T IL-12 SNP.

The present invention furthermore relates to the use of the inventive methods for predicting clinical response to a therapeutic compound in the treatment of ICAM-1 mediated schizophrenia.

The present invention finally relates to the manufacture and production of assays, kits, screens and therapeutics as in addition to antibodies to the protein ICAM-1. In particular, it relates to a method of screening for schizophrenia, comprising the step of:
(a) assaying a protein sample for the presence of the ICAM-1 protein having the 241A polymorphism,
wherein the presence of said polymorphism is indicative of a schizophrenia.

A candidate gene approach study investigating the G241A and A469G SNPs of the ICAM-1 gene, as well as the additional G1188T IL-12 SNP, is described in the following.

A total of 206 unrelated caucasian patients suffering from schizophrenia (117 male, 89 female; mean age 33.73±12.37 years, ranging from 17 to 71 years) were recruited at the Psychiatric Hospital of the University of Munich. Diagnoses were established according to the criteria of the DSM-IV by two independent experienced psychiatrists. Paranoid schizophrenia was diagnosed in 73.8% of the patients (DSM-IV: 295.3×), 13.2% carried a diagnosis of disorganised schizophrenia (DSM-IV: 295.1×), 2% of residual schizophrenia (DSM-IV: 295.6×). 1% was diagnosed as catatonic schizophrenics (DSM-IV: 295.2×), 5.8% as undifferentiated schizophrenics (DSM-IV: 295.9×), and 4.2% had a schizophreniform disorder (DSM-IV: 295.4×).

285 healthy caucasians from the general population (144 male, 141 female; mean age 41.5±11.3 years, ranging from 22 to 63 years), representing different social groups, were recruited as the control group. All controls were screened for past or present psychiatric illness and were medically examined including standard laboratory tests. History of psychiatric illness in a first degree relative was considered an exclusion criterion.

The study was approved by the local Ethics Committee, and all patients and controls gave their written informed consent after the aim of the study had been fully explained.

Genotyping

Genomic DNAs from all subjects were prepared from peripheral whole blood cells using the QlAamp system (Qiagen, Germany).

The genotyping of the two codons (G241A and A469G) of ICAM-1 was performed by the snapshot method using a PTC-200 thermo cycler (MJ Research, Watertown, Mass.) and a genetic analyser ABI Prism 310. In the first step the inventors carried out a multiplex PCR for both codons: forward primer (G241A): SEQ ID NO: 1, reverse primer (G241A): SEQ ID NO: 2; forward primer (A496G): SEQ ID NO: 6, reverse primer (A469G): SEQ ID NO: 7. The PCR was performed with 50 ng DNA in a total volume of 25 μl containing 0.25 μl AmpliTaqGold, 2.5 μl 10×PCR mix, 0.625 μl each primer and 2.5 μl dNTPs for 29 cycles of denaturation (95° C.), annealing (58° C.) and extension (72° C.). After restriction with SAP and Exo 1, a SNAPshot PCR for both codons was made. The SNAPshot primer ICAM4-S SEQ ID NO: 3 (G241A) and ICAM6-S SEQ ID NO: 8 (A469G) were used. The PCR was performed in a total volume of 10 μl containing 1,0 μl template and 1 μl SNAPshot primer and 5.0 μl SNAPshotReadyReaction Premix; annealing temperature 60° C. Afterwards, a restriction was performed and the SNAPshot PCR products were sequenced at the ABI Prism 310 genetic Analyser.

Statistical Analysis

Statistical analyses were performed using SPSS for windows (Version 10.0.7). Results are reported as means±SD. Genotype and allele frequencies of patients vs. controls, as well as between diagnostic subgroups were calculated using Chi² test. The relationship between genotype and age of onset was established by ANOVA test. A p value of 0.05 was considered significant. Together with genotype distribution, significances for allele frequencies are reported.

The inventors used case-control analysis to determine whether the ICAM-1 gene polymorphisms G241A and A469G are associated with schizophrenia. Homozygosity for the wildtype G241 allele was found in 224 control persons and 147 schizophrenic patients, while heterozygosity for the polymorphic allele was present in 57 control persons and in 56 patients. The rare homozygosity for the polymorphic A-allele was found in 4 controls and 3 patients. A statistical comparison of the genotype distribution did not reveal a significant difference between the two investigated groups (Chi²=3.513, df=2, p=0.173). Table 1 gives the genotype distribution in case control samples for the G241A polymorphism.

TABLE 1
Genotype count for the ICAM-1 G241A polymorphism in
schizophrenic patients (n = 206) and controls (n = 285).
Genotypes

| Phenotypes | G/G | G/A | A/A | p |
|---|---|---|---|---|
| Schizophrenics | 147 | 56 | 3 | 0.173 |
| Controls | 224 | 57 | 4 | |

However, the applicant further investigated the genotype distribution with regard to the gender of the tested patients and control subjects.

The most robust and consistently replicated findings in gender-differences literature on schizophrenia seem to be that, compared with women, men have an earlier age of illness onset, poorer course and outcome, fewer affective symptoms, poorer premorbid functioning, a greater history of obstetric complications in their mothers, and a lower familial loading for schizophrenia and affective disorder (reviewed by Salem and Kring, 1998). Accordingly, researchers have suggested that there are two sub-types of schizophrenia for which men and women are at different risks.

While there was no association between the A469G SNP neither with male, nor with female schizophrenic patients (data not shown), we found a markedly higher frequency of the polymorphic A allele in male schizophrenic patients compared to healthy male persons. This difference was not apparent in female patients.

It was found that in male schizophrenic patients, the A-allele was significantly more frequent than in male controls (31% vs. 18%, Chi²=5.761, df=1, p=0.016). This striking difference provides the basis for the method and kit for screening for schizophrenia according to the present invention. Table 2 gives the genotype distribution in male case control samples for the G241A polymorphism.

TABLE 2
Genotype count for the ICAM-1 G241A polymorphism in
male schizophrenic patients (n = 117) and
male controls (n = 144).
Genotypes

| Phenotypes | G/G | G/A; A/A | p |
|---|---|---|---|
| Schizophrenics | 81 | 36 (31%) | 0.016 |
| Controls | 118 | 26 (18%) | |

Because of the low frequency of homozygosity for the polymorphic A allele, homozygosity for the wildtype (GG) was compared with presence of the polymorphic allele (GA or AA).

Further investigations revealed that female schizophrenic patients exhibited substantially identical genotype distributions than the female control group (Chi²=0.001, df=1, p=0.976).

In sum, we found a marked association of the polymorphic ICAM-1 241A-allele with male schizophrenic patients, while female patients did not differ in their genotype distribution from the female control group. These results strongly indicate that gender differences in schizophrenia reflect differential risks for different subtypes of the disorder.

A further study dealt with the ICAM A469G polymorphism. Thirty-six of the 160 control persons were homozygous for the A-allele of the ICAM A469G polymorphism, while 92 were heterozygous and 32 showed homozygosity for the G-allele. Schizophrenic patients (160) exhibited a similar genotype distribution with 42 homozygous A-allele carriers, 86 heterozygous, and 32 being homozygous for the G-allele (Chi²=0.664, df=2, p=0.718). Table 3 gives the allele distribution and allele frequency in case control samples for the A469G polymorphism.

TABLE 3
Allele frequencies and genotypes count for the ICAM-1 A469G
polymorphism in schizophrenic patients (n = 160) and
controls (n = 160)

| | Genotyoes | | | | Allele | | |
|---|---|---|---|---|---|---|---|
| Phenotypes | A/A | A/G | G/G | p | A | G | p |
| Schizophrenics | 42 | 86 | 32 | .718 | 0.531 | 0.469 | .692 |
| Controls | 36 | 92 | 32 | | 0.513 | 0.488 | |

Hardy-Weinberg equilibrium was fulfilled regarding both polymorphisms in the population of healthy control persons.

There was no association between age of onset and genotype distribution among patients. Clinical subtypes of schizophrenia were not associated with a distinct geno-type of either polymorphism.

The inventors investigated ICAM-1 as one possible candidate gene in a schizophrenia based on suggestions that cellular immune activity may be altered in schizophrenia. We report here, for the first time, strong evidence of possible involvement of ICAM-1 gene G241A polymorphism in schizophrenia genetic susceptibility in male patients, whereas the A469G polymorphism was similarly distributed in patients and controls.

Further evidence of the usefulness of the G241A polymorphism are studies performed with the gene expression product and data that support the finding that the modified protein is a useful marker for schizophrenia. The protein itself may be useful in the manufacture and production of assays, kits, screens and therapeutics as in addition to antibodies to the protein all made using procedures well known to those skilled in the art.

The complete amino acid sequence of the ICAM-1 protein is published in the EMBL Protein Bank under accession number X59286 (see also: http://www.ncbi.nlm.nih.gov:80/entrez/query.fcgi?cmd=Retrieve&db=protein&list_ui ds=825682&dopt=GenPept). The soluble form of ICAM-1 (sICAM-1) is probably generated by alternative splice donor site selection. A 19-base deletion occurs right upstream of the transmembrane region giving rise to reading frameshift and eliminating the entire transmembrane and cytoplasmic domains, resulting in incapability of ICAM-1 molecules to reside in the membrane (Wakatsuki,T., Kimura,K., Kimura,F., Shinomiya,N., Ohtsubo,M., Ishizawa,M., and Yamamoto,M. (1995). A distinct mRNA encoding a soluble form of ICAM-1 molecule expressed in human tissues. Cell Adhes. Commun. 3, 283-292).

The following example is provided as evidence of the usefulness and ability to monitor soluble ICAM-1 in schizophrenia.

A total of 71 Caucasian patients suffering from schizophrenia (since at least 1 week without medication, 42 male and 29 female patients; mean age 31.4±10.6 years) were recruited at the Psychiatric Hospital of the University of Munich including 21 patients from a previous study about sICAM-1 in unmedicated and medicated schizophrenic patients (Schwarz,M. I., Riedel,M., Ackenheil,M., and Müller,N. (2000). Decreased Levels of Soluble Intercellular Adhesion Molecule-1 (sICAM-1) in Unmedicated and Medicated Schizophrenic Patients. Biol. Psychiat. 47, 29-33) who were re-investigated for the genetic G241A-polymorphism.

This study dealt with an investigation of a possible association between the G241A Polymorphism and sICAM-1 serum levels, recruiting only patients with a more than two months wash out period.

131 healthy Caucasians from the general population (72 male, 59 female; mean age 30.0±9.6 years), representing different social groups, were recruited as the control group. All controls were screened for past or present psychiatric illness and were medically examined including standard laboratory tests. History of psychiatric illness in a first degree relative was considered an exclusion criterion.

The study was approved by the local Ethics Committee, and all patients and controls gave their written informed consent after the aim of the study had been fully explained.

The sICAM-1 was estimated by a commercially available double-sandwich ELISA (Endogen, USA). Intra- and inter-assay variances were below 8%. Measurement was done in duplicates. The paired samples of one patient were always measured within one assay together with a set of control samples. The laboratory personnel were blinded regarding the source of the samples.

Statistical analyses were performed using SPSS for windows. Results are reported as the mean±SD. We used the t-test for independent samples and the Chi² test.

The results of the study are as follows:

The serum concentrations of sICAM-1 in schizophrenic patients were significantly reduced compared with those of the control group (schizophrenic patients: 302.5+105.3 ng/mL sICAM-1; controls: 338.8+79.8 ng/mL; two tailed t=5.12?, p=0.013; see FIGURE). Furthermore, a significant difference in the sICAM-1 serum levels was observed between female schizophrenic patients and female controls (n=29, 293.6±99.3 ng/ml versus n=59; 331.7±87.8 ng/ml; p=0.023). However, there was no similar relationship for the male subgroup (42 male patients: 315.5±108.6 ng/mL; 72 male controls: 344.5±72.8 ng/mL; p=0.128) .

We report here, for the first time, strong evidence of a functional immunological involvement of the ICAM-1 gene G241A polymorphism. Schizophrenic patients showed significantly reduced soluble ICAM-1 serum levels compared to healthy controls, in particular wirh respect to female patients and controls.

The G241A polymorphism leads to an nonsynonymous nucleotide exchange that results in a nonconservative amino acid replacement. Moreover, this occurs in the binding domain for MAC-1, a region that is important for the adhesion and activation of macrophages and monocytes. Thus, the affinity of ICAM-1 to MAC-1 may be reduced and the elevated sICAM-1 levels may reflect the increased expression of membrane-bound ICAM-1. This hypothesis is consistent with the significantly increased frequency of the 241 A-allele in Th1/immune-related diseases like rheumatoid arthritis (RA) (Macchioni,P., Boiardi,L., Casali,B., Nicoli,D., Farnetti,E., and Salvarani,C. (2000). Intercellular adhesion molecule 1 (ICAM-1) gene polymorphisms in Italian patients with rheumatoid arthritis. Clin. Exp. Rheumatol. 18, 553-558), Behcet's disease (Boiardi,L., Salvarani,C., Casali,B., Olivieri,I., Ciancio,G., Cantini,F., Salvi,F., Malatesta,R., Govoni,M., Trotta,F., Filippini,D., Paolazzi,G., Nicoli,D., Farnetti,E., and Macchioni,L. (2001). Intercellular adhesion molecule-1 gene polymorphisms in Behcet's Disease. ]. Rheumatol. 28, 1283-1287), giant cell arteritis (Salvarani,C., Casali,B., Boiardi,L., Ranzi,A., Macchioni,P., Nicoli,D., Farnetti,E., Brini,M., and Portioli,I. (2000). Intercellular adhesion molecule 1 gene polymorphisms n polymyalgia rheumatica/giant cell arteritis: association with disease risk and severity. J. Rheumatol. 27, 1215-1221) and ulcerative colitis (Braun,C., Zahn,R., Martin,K., Albert,E., and Folwaczny,C. (2001). Polymorphisms of the ICAM-1 gene are associated with inflammatory bowel disease, regardless of the p-ANCA status. Clin. Immunol. 101, 357-360). Moreover, elevated sICAM-1 serum levels have been described in all of these disorders (Aydintug,A. O., Tokgoz,G., Ozoran,K., Duzgun,N., Gurler,A., and Tutkak,H. (1995). Elevated levels of soluble intercellular adhesion molecule-1 correlate with disease activity in Behcet's disease. Rheumatol. Int. 15, 75-78; Coll-Vinent,B., Vilardell,C., Font,C., Oristrell,]., Hernandez-Rodriguez,J., Yague,J., Urbano-Marquez,A., Grau,J.M ., and Cid,M. C. (1999). Circulating soluble adhesion molecules in patients with giant cell arteritis. Correlation between soluble intercellular adhesion molecule-1 (sICAM-1) concentrations and disease activity. Ann. Rheum. Dis. 58, 189-192, 1999; Goke,M., Hoffmann,J. C., Evers,]., Kruger,H., and Manns,M. P. (1997). Elevated serum concentrations of soluble selectin and immunoglobulin type adhesion molecules in patients with inflammatory bowel disease. J. Gastroenterol. 32, 480-486; Littler,A. J., Buckley,C. D., Wordsworth,P., Collins,I., Martinson,J., and Simmons,D. L. (1997). A distinct profile of six soluble adhesion molecules (ICAM-1, ICAM-3, VCAM-1, E-selectin, L-selectin and P-selectin) in rheumatoid arthritis. Br. J. Rheumatol. 36, 164-169). However, the direct correlation between ICAM-1 genotype and sICAM serum levels have not been investigated in these disorders until now.

Schizophrenia, as well as the above described rheumatoid disorders are supposed to be of polygenic or oligogenic mode of transmission (McGue,M. and Gottesman,I. I. (1989). A single dominant gene still cannot account for the transmission of schizophrenia. Arch. Gen. Psychiatry 46, 478-480; McGuffin,P., Owen,M. J., and Farmer,A. E. (1995). Genetic basis of schizophrenia. Lancet 346 678-682). Thus, the interaction of several susceptibility genes with the ICAM-1 G241A polymorphism may be responsible for the biochemical effect of the herein investigated ICAM-1 polymorphism in schizophrenia and rheumatoid disorders, while this effect may be compensated in healthy persons.

Altogether, our finding represents the first biochemical evidence for the functionality of the ICAM-1 G241A polymorphism.

Without intending to establish a certain theory as explanation for the observed association between the G241A polymorphism and the susceptibility to schizophrenia, the following mechanisms of action are taken into consideration.

The genotype distribution of the herein investigated schizophrenic patient group is similar to that observed in the above cited groups of patients with immune mediated disorders with a nearly two-fold frequency of the polymorphic A allele in schizophrenic patients, as compared to the healthy control group. Thus, our finding of the altered ICAM-1 G241A genotype distribution, particularly in the male subgroup, supports the hypothesis of an altered cellular immune function in schizophrenia.

As recently reviewed by Rothermund and colleagues, a marked decrease of the Th1 cellular immune function is a convincing result in schizophrenia research (Rothermundt M, Arolt V, Bayer T A. Review of immunological and immunopathological findings in schizophrenia. Brain Behav Immun 2001; 15: 319-339), whereas schizophrenic patients seem to be predesposed toward more vigorous B-cell responses (Printz D J, Strauss D H, Goetz R, Sadiq S, Malaspina D, Krolewski J, Gorman J M. Elevation of CD5+B lymphocytes in schizophrenia. Biol Psychiatry 1999; 46: 110-118; McAllister C G, Rapaport M H, Pickar D, Podruchny T A, Christison G, Alphs L D, Paul S M. Increased numbers of CD5+B lymphocytes in schizophrenic patients. Arch Gen Psychiatry 1989; 46: 890-894).

This immune alteration may be based on a genetic predisposition influencing the Th1/Th2 balance. Moreover, this genotype could also predispose for an altered anti-viral immune response. This is of particular interest as a neurodevelopmental disturbance after a prenatal virus infection during brain maturation has been proposed (Marenco S, Weinberger D R. The neurodevelopmental hypothesis of schizophrenia: following a trail of evidence from cradle to grave. Dev Psychopathol 2000; 12: 501-527; Yolken R H, Karlsson H, Yee F, Johnston-Wilson N L, Torrey E F. Endogenous retroviruses and schizophrenia. Brain Res Rev 2000; 31: 193-199). A genetic factor, influencing the anti-viral defence reaction of the fetus, would link the hereditary and the environmental component of schizophrenia. The cell adhesion molecule ICAM-1 is one of those factors, contributing to antigen presentation and activation of the cellular immune reaction during viral infections (Marker O, Scheynius A, Christensen J P, Thomsen A R. Virus-activated T cells regulate expression of adhesion molecules on endothelial cells in sites of infection. J Neuroimmunol 1995; 62: 35-42). Th1 cells need costimulation for the secretion of IL-2 as well as for enhancing the secretion of IFN-_{X}, while Th2 cells efficiently produce IL-4 even in the absence of costimulation (Croft M, Dubey C. Accessory molecule and costimulation requirements for CD4 T cell response. Crit Rev Immunol 1997; 17: 89-118). Thus, a deficiency of ICAM-1 activity may induce a bias towards a Th2 predominance. Indeed, it was demonstrated that Th2 cells are able to diminish proinflammatory reactions in the CNS (Gimsa U, Wolf S A, Haas D, Bechmann I, Nitsch R. Th2 cells support intrinsic anti-inflammatory properties of the brain. J Neuroimmunol 2001; 119: 73-80).

This is consistent with neuropathological studies of schizophrenic brains, where a lack of gliosis or lymphocytic infiltrates in regions of neuronal cell loss was demonstrated (Roberts G W, Colter N, Lofthouse R, Bogerts B, Zech M, Crow T J. Gliosis in schizophrenia: a survey. Biol Psychiatry 1986; 21: 1043-1050; Arnold S E. Neurodevelopmental abnormalities in schizophrenia: insights from neuropathology. Dev Psychopathol 1999; 11:439-456; Pearce B D. Schizophrenia and viral infection during neurodevelopment: a focus on mechanism. Mol Psychiatry 2001; 6: 634-646). Thus, a proposed virus-induced neurodevelopmental disruption as cause of this cell loss appears to have occurred without a marked inflammatory process. Following this line of evidence, a genetic variation of the ICAM-1 protein, leading to a reduced function, may be one of the links between heredity and environmental cause of schizophrenia.

Altogether, the association of the A241 polymorphic allele with several autoimmune diseases and schizophrenia—all of them described to be of polygenic nature (Chernajovsky Y, Dreja H, Daly G, Annenkov A, Gould D, Adams G, Croxford J L, Baker D, Podhajcer O L, Mageed R A. Immuno- and genetic therapy in autoimmune diseases. Genes Immun 2000; 1: 295-307)—gives evidence for this polymorphism as being one of the contributing genetic risk factors. The diverse combination with additional susceptibility genes and environmental factors may then lead to the different phenotypes of the respective disorders.

In the following, the experimental methods and results relating to the further SNP suitable as markers for schizophrenia will be discussed.

IL-12 p40 G1188T SNP (untranslated) (chr. 5q31.1-33.1)

The sequence (mRNA) of the IL-12 coding region is described in the following references:

http://www.ncbi.nlm.nih.gov:80/entrez/query.fcgi?cmd=Retrieve&db=nucleotide&list_uids=5923854&dopt=GenBank (AF180563); however, the complete sequence of the IL-12 gene including exons and introns is not yet available. OMIM description: http://www.ncbi.nim.nih.gov:80/entrez/dispomim.cgi?cmd=entry&id=161561

Genotyping

The genotyping of the IL-12 G1188T SNP was performed by the snapshot method using a PTC-200 thermo cycler (MJ Research, Watertown, Mass.) and a genetic analyser ABI Prism 310. In the first step a PCR was carried out for both codons: forward primer IL-12: SEQ ID NO: 4; reverse primer IL-12: SEQ ID NO: 5. The PCR was performed with 50 ng DNA in a total volume of 10 μl containing 0,1 μl AmpliTaqGold, 1 μl 10×PCR mix, 0,2 μl each primer and 1 μl dNTPs for 30 cycles of denaturation (95° C.), annealing (57° C.) and extension (72° C.). After restriction with SAP and Exo I, a SNAPshot PCR for both codons was made. The SNAPshot primer was SEQ ID NO: 9. The PCR was performed in a total volume of 10 μl containing 2 μl template and 1 μl SNAPshot primer and 2,5 μl SNAPshotReadyReaction Premix; annealing temperature 60° C. Afterwards, a restriction was performed and the SNAPshot PCR products were sequenced at the ABI Prism 310 genetic Analyser (Applied Biosystems, Foster City, Calif.).

Results

In the study 152 schizophrenic patients and 221 healthy control individuals were included for the IL-12 p40 G1188T polymorphism. Genomic DNA was isolated from whole blood according to standard procedures. 6 of the control persons were homozygous for the G-aliele of the IL-12 G1188T polymorphism, while 74 were heterozygous and 141 showed homozygosity for the T-allele. Schizophrenic patients exhibited a genotype distribution with 4 homozygous G-allele carriers, 63 heterozygous, and 85 being homozygous for the T-allele (Chi²=2.48, df=2, p=0.289). Table 4 gives the allele distribution and allele frequency in case control samples for the IL-12 G1188T polymorphism. Although the IL-12 polymorphism was similar in patients and controls, a combination of the IL-12 polymorphism and the ICAM-1 polymorphism gave different frequencies for schizophrenic patients and controls.

TABLE 4
Allele frequency and genotype count for the IL-12 G1188T
polymorphism in schizophrenic patients (n = 152) and
controls (n = 221).

| | Genotypes | | | | Allele frequencies | | |
|---|---|---|---|---|---|---|---|
| Phenotypes | G/G | G/T | T/T | p | G | T | p |
| Schizophrenics | 4 | 63 | 85 | 0.289 | 71 | 233 | 233 |
| Controls | 6 | 74 | 141 | | 86 | 356 | |

Obviously, many modifications and variations of the present invention are possible in light of the above teachings. Thus, it is to be understood that, within the scope of the appended claims, the invention may be practiced otherwise than as specifically described above.

Summary of Sequences

SEQ ID NO: 1 is a nucleic acid sequence of a primer suitable for amplification of genomic nucleic acid encoding the A241 allele of the ICAM-1 gene (forward primer).

SEQ ID NO: 2 is a nucleic acid sequence of a primer suitable for amplification of genomic nucleic acid encoding the A241 allele of the ICAM-1 gene (reverse primer).

SEQ ID NO: 3 is a SNAPshot primer suitable for use in a SNAPshot PCR for the G241A codon of ICAM-1.

SEQ ID NO: 4 is a nucleic acid sequence of a primer suitable for amplification of genomic nucleic acid encoding the T1188 allele of the IL-12 protein (forward primer).

SEQ ID NO: 5 is a nucleic acid sequence of a primer suitable for amplification of genomic nucleic acid encoding the T1188 allele of the IL-12 protein (reverse primer).

SEQ ID NO: 6 is a nucleic acid sequence of a primer suitable for amplification of genomic nucleic acid encoding the G496 allele of the ICAM-1 gene (forward primer).

SEQ ID NO: 7 is a nucleic acid sequence of a primer suitable for amplification of genomic nucleic acid encoding the G496 allele of the ICAM-1 gene (reverse primer).

SEQ ID NO: 8 is a SNAPshot primer suitable for use in a SNAPshot PCR for the A496G codon of ICAM-1.

SEQ ID NO: 9 is a SNAPshot primer suitable for use in a SNAPshot PCR for the G1188T codon of IL-12.

1. A method of screening for schizophrenia, comprising the step of:
(a) assaying a nucleic acid sample for the presence of a codon encoding arginine at amino acid position 241 of the ICAM-1 protein,
wherein the presence of a codon encoding arginine at amino acid position 241 of the ICAM-1 protein is indicative of a schizophrenia.

2. The method according to claim 1, wherein the method further comprises the step of obtaining a nucleic acid sample prior to said step (a).

3. A method according to claim 1, wherein said assaying comprises amplification of the ICAM-1 gene or a suitable fragment thereof by a technique suitable therefore.

4. A method according to claim 3, wherein said amplification comprises polymerase chain reaction with the primers having the sequences SEQ ID NO: 1 and SEQ ID NO: 2.

5. A method according to claim 4, wherein said amplification further comprises a SNAPshot polymerase chain reaction with the SNAPshot primer having the sequence SEQ ID NO: 3.

6. A method according to claim 1, wherein the nucleic acid sample is isolated from blood cells.

7. A method according to claim 1, wherein the method further comprises assaying said sample for an additional marker associated with the susceptibility for schizophrenia, said marker being the G1188T IL-12 single nucleotide polymorphism.

8. A kit for screening for schizophrenia comprising reagents tailored to identify the polymorphism at amino acid position 241 of the ICAM-1 protein.

9. The kit according to claim 8, wherein said reagents comprise primers having the sequences SEQ ID NO: 1 and SEQ ID NO: 2 and reagents necessary to perform PCR.

10. The kit according to claim 9, wherein said reagents further comprise the SNAPshot primer having the sequence SEQ ID NO: 3.

11. The kit according to claim 8, wherein the kit further comprises reagents tailored to identify the G1188T IL-12 single nucleotide polymorphism.

12. The kit according to claim 11, wherein said reagents comprise primers selected from SEQ ID NO: 4 and SEQ ID NO: 5.

13. The method according to claim 1 for predicting clinical response to a therapeutic compound in the treatment of ICAM-1 mediated schizophrenia.

14. A method of screening for schizophrenia, comprising the step of:
(a) assaying a protein sample for the presence of the ICAM-1 protein having the 241A polymorphism, wherein the presence of said polymorphism is indicative of a schizophrenia.

15. A method according to claim 14, wherein said sample is a blood sample and the ICAM-1 protein is soluble.

16. A method according to claim 15, wherein comparative samples are obtained from healthy normal controls and used to compare to said samples taken from suspect individuals wherein lower serum levels of sICAM-1 protein is indicative of schizophrenia.

17. A method according to claim 16, wherein the protein sample is isolated from blood cells.

18. A method according to claim 17, wherein the method further comprises assaying said sample for an additional marker associated with the susceptibility for schizophrenia, said marker being the G1188T IL-12 single nucleotide polymorphism.

19. A blood kit for screening for schizophrenia comprising reagents tailored to identify the polymorphism at amino acid position 241 of the ICAM-1 protein.

20. The kit according to claim 19, wherein the kit further comprises reagents tailored to identify the G1188T IL-12 single nucleotide polymorphism.

21. The method according to claim 14 for predicting clinical response to a therapeutic compound in the treatment of ICAM-1 mediated schizophrenia.

22. An antibody specifically reactive to ICAM-1 G241 protein.

23. An antibody specifically reactive to ICAM-1241A protein.

24. An antibody according to claim 22 specifically reactive to an epitope containing the G241 amino acids of ICAM-1.

25. An antibody according to claim 23 specifically reactive to an epitope containing the 241A amino acids of ICAM-1.

26. A treatment for schizophrenia comprising measuring and detecting the presence of the 241A polymorphism in an individual or population followed by the administration of agents useful for the treatment of schizophrenia.


SEQUENCE LISTING (9 sequences)

SEQ ID NO: 1 - DNA, 20 bp, Artificial
  Primer derived from human ICAM-1 gene
actcccccacaacttgtcag

SEQ ID NO: 2 - DNA, 20 bp, Artificial
  Primer derived from human ICAM-1 gene (reverse primer)
gttccggagtcagtcacact

SEQ ID NO: 3 - DNA, 20 bp, Artificial
  SNAPshot primer for ICAM-1
ccgttgtctgttccctggac

SEQ ID NO: 4 - DNA, 22 bp, Artificial
  Primer derived from human IL-12 gene
aaagacacaacggaatagaccc

SEQ ID NO: 5 - DNA, 20 bp, Artificial
  Primer derived from human IL-12 gene (reverse primer)
cgttccgacgttcatgtagt

SEQ ID NO: 6 - DNA, 21 bp, Artificial
  Primer derived from human ICAM-1 gene
gagatcttgagggcacctacc

SEQ ID NO: 7 - DNA, 18 bp, Artificial
  Primer derived from human ICAM-1 gene (reverse primer)
cggtcgccttctagttct

SEQ ID NO: 8 - DNA, 21 bp, Artificial
  SNAPshot primer for ICAM-1
agagcacattcacggtcacct

SEQ ID NO: 9 - DNA, 26 bp, Artificial
  SNAPshot primer for IL-12
gattgtttcaatgagcatttagcatc